(19) United States
(12) Patent Application Publication
(10) Pub. No.: US 2011/0130542 A1
(43) Pub. Date: Jun. 2, 2011

(54) METHOD FOR THE SELECTIVE SEPARATION OF PEPTIDES AND PROTEINS BY MEANS OF A CRYSTALLIZATION PROCESS

(75) Inventors: Joerg Kauling, Koln (DE); Dirk Havekost, Koln (DE); Hans-Jürgen Henzler, Solingen (DE)

(73) Assignee: BAYER TECHNOLOGY SERVICES GMBH, Leverkusen (DE)

(21) Appl. No.: 12/999,193

(22) PCT Filed: Jun. 16, 2009
(86) PCT No.: PCT/EP09/04306

§ 371 Date: Dec. 15, 2010

(30) Foreign Application Priority Data
Jun. 23, 2008 (DE) 10 2008 029 401.2

(51) Int. Cl. C07K 1/30

(52) U.S. Cl. 530/344; 530/419

(57) ABSTRACT

Method for removing and selective separating peptides and proteins from a solution by controlled crystallization.

This is a 371 of PCT/EP2009/004306, filed 16 Jun. 2009 (international filing date), which claims foreign priority benefit under 35 U.S.C. §119 of German Patent Application No. 10 2008 029 401.2 filed Jun. 23, 2008.

The present invention relates to a method for crystallizing peptides and proteins. Methods for depositing and separating peptides or proteins play an important role, for example, in isolating peptides and proteins from body tissue of bacterial cell cultures or animal cell cultures. In the field of clinical use of proteins, there are only a few industrial methods (e.g., for lysozyme, insulin, Trasylol®) where deposition is performed as a batch process and subsequent recovery of proteins takes place by means of centrifugation or filtration.

### BACKGROUND OF THE INVENTION

It can be shown for a multiplicity of applications that conventional batch reactors, in which addition of precipitant and/or change in temperature to deposit a peptide/protein and also deposition itself and particle growth take place in a single stirred reactor, are not optimal for depositing peptides and proteins. The cause are inhomogeneities as a result of insufficient mixing. Supersaturation of the solution due to insufficient mixing leads to a reduction in product quality. On the other hand, there is a limit to intensifying mixing, since too intensive a mixing could result in too high a mechanical stress on the proteins/peptides deposited. Proteins/peptides may be destroyed.

The separability and the yield are increased by a uniform particle size and pure particles. Small particles having a uniform distribution of particle sizes are needed for producing pharmaceuticals in particular.

Separating various proteins/peptides from one another by selectively depositing only one protein/peptide is also difficult in batch reactors for the abovementioned reasons.

Therefore, the object is to provide a method for depositing and/or separating peptides and proteins which allows setting of controlled conditions for a multiplicity of applications in order to obtain high yield, high purity, and defined particle sizes having a very narrow distribution.

It was found that, surprisingly, this object is achieved on depositing proteins/peptides via a controlled crystallization where mixing of the peptide/protein solution with a crystallization agent and/or optional cooling/warming when crystallizing by cooling/warming and actual crystallization take place spatially separated from one another.

### SUMMARY OF THE INVENTION

The present invention, accordingly, provides a method for depositing and/or selectively recovering a peptide/protein from a solution which comprises at least the following steps:
a) mixing a protein/peptide solution with a crystallization agent,
b) optionally cooling or warming,
c) crystallizing a protein/peptide,
   wherein steps a) to c) proceed spatially separated from one another.

### DETAILED DESCRIPTION

Hereinafter, the term “peptides” will also be used to mean proteins. The term “peptides” will further be understood to mean substituted and unsubstituted peptides and/or proteins, where possible substituents can be, e.g., glycosides, nucleic acids, alkyl groups, aryl groups, and mixtures thereof. The substitutions can occur on the backbone of the peptide or on the side groups.

“Mixing” is understood to mean a process which serves the purpose of equalizing locally present concentration or temperature gradients between the components of the phases to be mixed. The goal is to achieve a very high homogeneity of the new material. This goal is achieved when a random sample from the mixture mirrors the ratio of the initial materials (materials to be mixed) with a defined accuracy. “Mixing” occurs at the macroscopic level by convection and at the molecular level as a result of diffusion. The process of mixing occurs in three substeps which take place both consecutively and simultaneously. In the first substep of macromixing, single subvolumes characterized by their concentration are distributed in the entire mixer by convective transport. Local fluctuations in concentration and also the extent of the subvolumes remain substantially unchanged. Only a deformation as a result of viscous friction takes place. In the second substep of macromixing, the dimensions of the subvolumes are reduced depending on the viscosity of the fluids, either by molecular or turbulent momentum exchange. The size of the subvolumes characterized by a homogenous concentration decreases to a threshold value. This value characterizes the transition from macromixing to micromixing.

Below this threshold size, the volume elements are not further dissipatable by turbulent fluctuation movements. Further equalization of concentration is caused by molecular diffusion alone. The macromixing procedure and micromixing procedure are each allocated a time constant. More specific details about micromixing and macromixing can be obtained from the literature, e.g., K. Kling, Visualisieren des Mikro- und Makromischens mit Hilfe zweier fluoreszierender und chemisch reagierender Farbstoffe (Visualizing micromixing and macromixing with the help of two fluorescent and chemically reactive dyes), thesis for the attainment of the academic degree Doctor of Engineering approved by the Faculty of Mechanical Engineering at the University of Hanover, 2004.

The term “spatially separated” means that steps a) to c) take place in different vessels (which are connected via one another via, e.g., pipes). The term “spatially separated” is, however, also to be understood to mean that steps a) to c) are carried out in different zones/sections of a vessel, e.g., in different sections of a tubular reactor.

The term “crystallization agent” is to be understood to mean any chemical compound or mixture of chemical compounds which causes or promotes expulsion of peptides in the form of crystals from a solution, more particularly from an aqueous solution. In a preferred embodiment of the present invention, the crystallization agent comprises at least one compound from the following group: peptides, proteins, ethanol, salt solutions, acids, pH buffers, phenol, nonionic polymers, ionic polyelectrolytes.

“Crystallization” must be distinguished from precipitation. Crystallization is understood to mean the process in which peptides nucleate under controlled conditions, i.e., form crystals which grow in a controlled manner. The result of a crystallization are crystals having a defined morphology. Furthermore, crystallized peptides show a narrower particle size distribution than precipitated peptides. Crystallization is generally a slower process than precipitation.

Precipitation is understood to mean the process in which peptides are deposited in a fast process from a solution by adding a precipitant and/or as a result of temperature change. The result of a precipitation is a deposit which is hereinafter termed a precipitate. A precipitate has a broad particle size distribution. A large fraction of the particles are amorphous and/or polymorphous (not uniformly crystalline). The precipitate contains inclusions of solvent and precipitant and is therefore less pure than the result of a crystallization. The precipitate may be gel-like and difficult to filter. While precipitation is simple to accomplish by adding a precipitant in excess, crystallization requires controlled conditions under which crystals can form and grow. Crystallization is technically more complicated than precipitation. Crystallization and precipitation are subsumed hereinafter under the term deposition.

### BRIEF DESCRIPTION OF THE DRAWINGS

FIG. 1 shows a schematic illustration of a device for carrying out the method according to the invention in a preferred embodiment.

FIG. 2 shows schematically the solubility curve of a peptide and the mode of operation involving two deposition variants, viz., fed-back mode B and batch mode A.

FIG. 3 shows the solubility curves of two peptides, P1 and P2, in one diagram.

FIG. 4 illustrates a preferred mixing element for step a) of the method according to the invention.

FIG. 5 shows a preferred embodiment of a device for carrying out the method according to the invention.

FIG. 6 shows a variant of the device shown in FIG. 5 for performing the method according to the invention.

FIG. 7 shows schematically the solubility ratios of an aqueous lysozyme solution.

FIG. 8 shows a further embodiment of a device for carrying out the method according to the invention.

Step a) of the method according to the invention is carried out in a mixing element. In a preferred embodiment of the method according to the invention, step a) is carried out in a jet mixer having at least two inlets, where one of the inlets is intended for introducing the peptide solution and a second inlet is intended for introducing the precipitant. At the downstream end of the mixing element is an outlet. Between the inlets and the outlet are the mixing chamber and an orifice plate. Such a construction enables a very good commixing of the streams, even at a very small throughput ratio q₂/q₁, where q₁ and q₂ are the streams through inlets 1 and 2.

In a preferred embodiment of the method according to the invention, the macroscopic mixing time t_{Ms} in step a) is 1 ms≦t_{Ms}≦1000 ms; in an especially preferred embodiment, the mixing time in step a) is 10 ms≦t_{Ms}≦100 ms.

In a preferred embodiment of the method according to the invention, the average mixing speed v (average mixing speed within the mixing chamber) in step a) is 0.05 m/s≦v≦5 m/s. As a result, the time for step a) is kept as short as possible. In a preferred embodiment, the mixing speed in step a) is 0.2 m/s≦v≦1.5 m/s, especially preferably 0.3 m/s≦v≦1 m/s.

In a preferred embodiment of the method according to the invention, the pressure drop Δp across the mixing element in step a) is 0.05 bar≦Δp≦20 bar. The pressure drop is preferably 0.1 bar≦Δp≦2.5 bar, especially preferably 0.2 bar≦Δp≦1 bar.

The ratio of d₁ (diameter of inlet 1 for the peptide solution) to Dₛ (width of the mixing chamber) is preferably 0.1≦d₁/Dₛ≦0.4, especially preferably 0.2≦d₁/Dₛ≦0.3. The ratio of d₂ (diameter of inlet 2 for the precipitant) to Dₛ (width of the mixing chamber) is preferably 0.05≦d₂/Dₛ≦0.3, especially preferably 0.08≦d₂/Dₛ≦0.13.

The size of the mixing chamber (Dₛ) is chosen such that turbulent stream conditions prevale. The diameter ratio d₁/d₂ is preferably chosen, depending on the flow rates q₁/q₂, such that the momenta of the colliding streams are approximately the same.

In a preferred embodiment of the method according to the invention in which crystallization is induced or supported by means of cooling or warming, an in-line heat exchanger is used in step b) for cooling or warming Preferably, a helically coiled heat exchanger is used, since it provides very good heat transfer and is simple to clean.

In a preferred embodiment of the method according to the invention, the mixture is continuously stirred during step c). It is preferred to use for stirring at least one impeller which causes only minimal mechanical stress to the particles. It is preferred to use an impeller having a larger diameter where the blades are preferably arranged radially so that mainly a radial stream results. It is preferred to use blade impellers in which the blades are fixed to a common axle, have various radial orientations, and exhibit little, if any, vertical slant. The number z of the blades is preferably 3≦z≦9, especially preferably 4≦z≦6. The stirring speed is preferably close to the point at which the crystals formed are just suspending.

In a preferred embodiment of the method according to the invention, the stirred vessel is equipped with baffles, e.g., with four baffles having a width of 0.1 D, where D is the diameter of the vessel or vessel section in which step c) is performed. It is also possible to place the stirrer eccentrically, in which case the eccentricity e/D is preferably 0≦e/D≦0.15, where e is the distance between the stirrer outer edge and the wall of the vessel or vessel section in which step c) is performed. The mixing quality of the stirrer is influenced advantageously by this embodiment for a multiplicity of applications. Inter alia, the cleanability of the crystallization vessel is improved by using an eccentric stirrer.

In a preferred embodiment of the method according to the invention, the ratio of stiffing blade diameter d to the diameter D of the vessel or vessel section in which step c) is carried out is 0.4≦d/D≦0.7. As a result, minimal particle stress is achieved. The ratio is preferably in the range of 0.45≦d/D≦0.65, especially preferably in the range of 0.5≦d/D≦0.6.

The ratio of stirring blade height h to stirring blade diameter d is in the range of 0.15≦h/d≦1.3.

When using an impeller system having two or more impellers, the ratio h/d for all impellers is in the range of 0.25≦h/d≦0.25. Especially preferably, all impellers have the same dimensions.

In a preferred embodiment of the method according to the invention, the ratio between the volume of the vessel or vessel section in which step a) is carried out and the volume of the vessel or vessel section in which step c) is carried out is greater than or equal to 0.01 and smaller than or equal to 0.1. It was found that, surprisingly, it can be advantageous for a multiplicity of applications to use a small mixing volume in proportion to the crystallization volume, since by this means the precipitant in step a) can be present in a greater excess without uncontrolled deposition occurring.

In a preferred embodiment of the method according to the invention, the ratio between the volume of the vessel or vessel section in which step a) is carried out and the volume of the vessel or vessel section in which step b) is carried out is greater than or equal to 0.02 and smaller than or equal to 0.08.

Owing to steps a) and b), step c) takes place in a controlled manner. Step c) preferably takes place automatically by carrying out steps a) and b), i.e., preferably no external stimuli are necessary in order to induce crystallization. It is preferable to simply stir in order to maintain homogenous conditions, and time is allowed for crystals to form and grow.

Deposition and/or recovery of a peptide from solution takes place according to the invention by crystallization. In a preferred embodiment of the method according to the invention, depositing and/or recovering a peptide from solution takes place by adding a crystallization agent stepwise along the solubility curve of the peptide. Crystallization agent is added always stepwise at an amount such that the solution supersaturates with the peptide to be removed and the peptide therefore crystallizes out. Preferably, only a slight excess of crystallization agent is added in each step in order to prevent the uncontrolled precipitation of the peptide. According to the invention, the mixing of peptide solution and crystallization agent takes place spatially separated from the actual crystallization.

In a further embodiment of the method according to the invention, depositing and/or recovering a peptide from solution takes place by stepwise warming or cooling, i.e., by raising or lowering the temperature stepwise, depending on whether the crystallization is promoted/induced by warming or cooling. The temperature change takes place along the solubility curve of the peptide: the temperature is changed stepwise to such an extent that the solution supersaturates with the peptide to be removed, and so the peptide crystallizes out. Preferably, the temperature is changed in small steps in order to prevent the uncontrolled precipitation of the peptide. According to the invention, the temperature change takes place spatially separated from the actual crystallization.

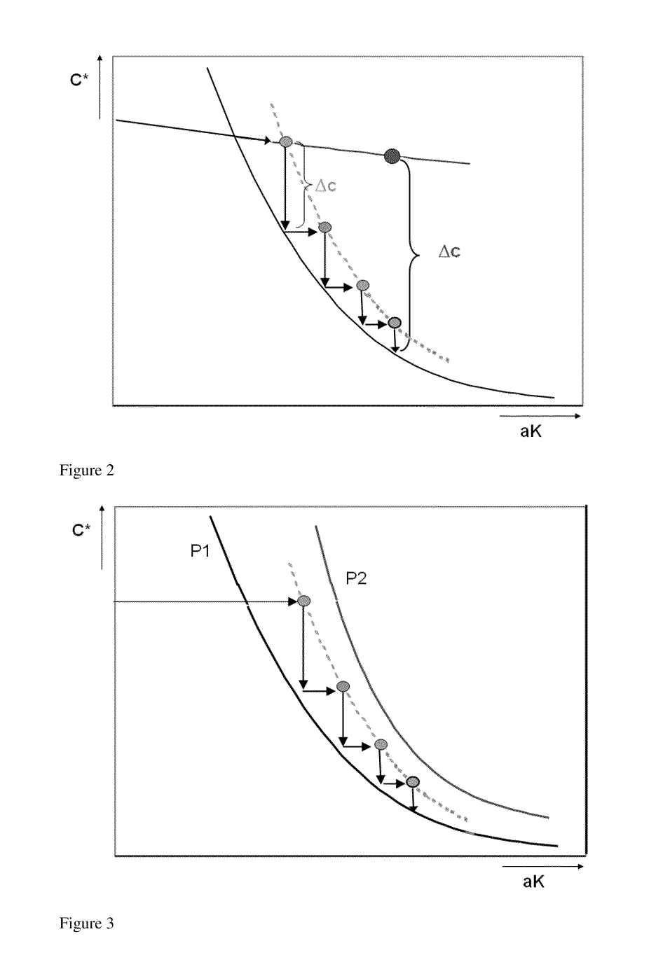

Examples of solubility curves are given in FIGS. 2, 3, and 7. The solubility curve of a peptide can be determined empirically (see, e.g., example 1). The concentration of dissolved peptide can take place, e.g., gravimetrically by evaporating a defined amount of solution and weighing out the remaining peptide, spectrometrically, or by other established methods for determining concentration which are known to a person skilled in the art.

In a preferred embodiment, the method according to the invention, accordingly, further comprises step d) after steps a) and c) or a), b), and c):
d) adding a portion of the solution of the crystallization suspension from step c) to the mixture in step a) or to the mixture in step b) when crystallizing by cooling or warming.

Step d) can take place continuously or discontinuously. Through the additional introduction of step d), the crystallization can be carried out continuously or discontinuously, and improves for a series of applications the crystallization conditions, resulting in improved product quality.

Step d) is preferably carried out in a mixing chamber in which the various mixtures/solutions are brought together.

In a preferred embodiment, the method according to the invention comprises step a₁) and a₂) after steps a) and c) or a), b), and c):
a₁) admixing further crystallization agent
a₂) optionally repeating steps a₁) and a₂).

Step a₁) is preferably carried out in a mixing chamber in which the various mixtures/solutions are brought together.

The invention is elucidated in detail below by way of example with the help of the figures, without, however, restricting the invention to these figures.

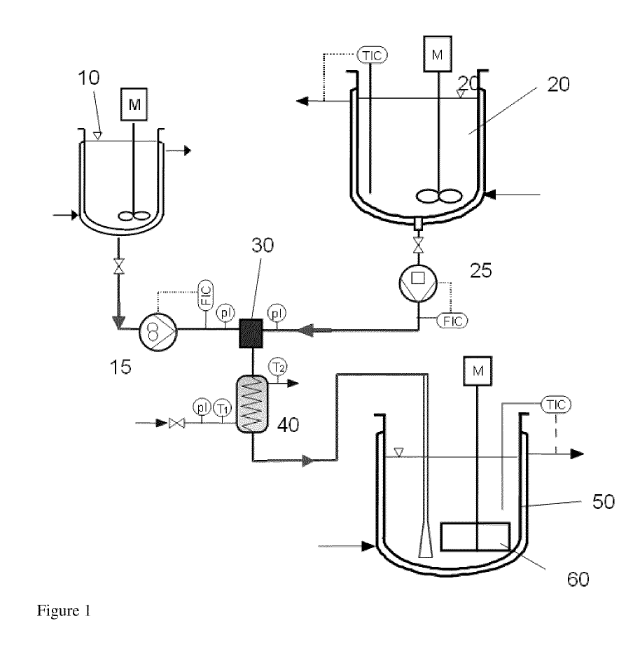

FIG. 1 shows a schematic illustration of a device for carrying out the method according to the invention in a preferred embodiment. The device comprises a vessel 10 which serves as a receiver for crystallization agent, a vessel 20 which serves as a receiver for peptide solution, a mixing element 30, a heat exchanger 40, and a vessel 50 for crystallization. The vessels 10 and 20 have a stirrer. Vessel 10 is connected to the mixing element 30 via a first pump 15. Vessel 20 is also connected to the mixing element 30 via a second pump 25. Step a) of the method according to the invention is performed in mixing element 30. When crystallizing by cooling or warming, the temperature of the mixture is changed by means of heat exchanger 40 and the mixture is introduced into the vessel 50 for crystallization. In a preferred embodiment, the pipe through which the mixture is introduced into the vessel 50 has a funnel-shaped design, as illustrated schematically in FIG. 1. The opening angle α of the funnel is in the range of 2°≦α≦8°. A blade stirrer is arranged eccentrically in the vessel 50.

FIG. 2 shows schematically the solubility curve of a peptide and the mode of operation involving two deposition variants, viz., fed-back mode B and batch mode A.

In the diagram, the concentration c* of a peptide in solution is plotted against the amount of crystallization agent aK which has been added to the solution. With increasing amount of crystallization agent aK, the concentration c* of dissolved peptide decreases, since a portion of the peptide amount is brought to crystallization by the crystallization agent and thus expelled from the solution. In the figure, two possible deposition processes are illustrated. In the case of process A, a large amount of crystallization agent is added once. The amount of crystallization agent added is to the right of the solubility curve in the diagram of FIG. 2, and so peptide should be precipitated. Through the sudden addition of the crystallization agent, the peptide solution is supersaturated with peptide. The peptide is rapidly deposited.

Through process B, a controlled crystallization is possible. In the case of process B, the same amount of crystallization agent is added as in the case of process A, but in smaller doses which are added one after the other with a time interval between doses. It is preferred to move along the solubility curve c*, i.e., only a slight excess of crystallization agent is always added. In a first addition of crystallization agent, the peptide solution becomes only slightly supersaturated. Peptide is deposited and the concentration of dissolved peptide sinks (Δc) to a concentration which is again on the solubility curve. Crystallization agent is added again, the solution is supersaturated with peptide, and peptide is deposited (Δc). The peptide concentration of the solution sinks to a value on the solubility curve, and so on. Through the stepwise addition of crystallization agent in small doses, controlled crystallization conditions are created. Only a small supersaturation Δc/c* of the solution takes place in each step. The peptides have time for crystallization and for crystal growth. The peptide deposited has a defined form and composition and consists of crystals which have a narrow particle size distribution. The crystallization process is preferably supported by stirring and/or temperature control. Instead of by adding crystallization agent, the peptide can also be deposited by controlled warming or cooling. In this case, the x-axis would not indicate the amount of crystallization agent aK added, but the increase or decrease in temperature T. Fed-back mode B is a preferred embodiment of the method according to the invention, wherein the mixing of peptide solution/suspension with crystallization agent and the crystallization itself take place according to the invention in separate vessels or vessel sections.

The controlled process B, in which only a slight supersaturation Δc/c* of the solution takes place stepwise, has the following advantages over process A in a multiplicity of applications:
prevention of uncontrolled nucleation,
through variation of the ratio Δc/c*, the ratio of particle growth to nucleation rate can be influenced and the crystallization result thus improved,
generation of larger crystals with a narrower particle size distribution,
peptides can be selectively crystallized from peptide mixtures (see, e.g., FIG. 3)
less incorporation of water and lower inclusion of foreign materials in the deposition product,
lower tendency to form polymorphous deposits,
avoidance of precipitate,
purer products, since coprecipitation is avoidable,
increased reproducibility.

FIG. 3 shows the solubility curves of two peptides, P1 and P2, in one diagram. In the diagram, the concentrations c* of the peptides P1 and P2 in solution are plotted against the amount of crystallization agent aK added. FIG. 3 schematically illustrates that peptide P1 can be selectively deposited from the solution by controlled addition of crystallization agent and controlled crystallization, while peptide P2 remains completely in solution. If the amount of crystallization agent being added stepwise in FIG. 3 were to be added to the solution at once, then peptides P1 and P2 would be expelled together and a separation would not be possible. Instead of by adding crystallization agent, a peptide can also be selectively deposited by controlled warming or cooling. In this case, the x-axis would not indicate the amount of crystallization agent aK added, but the increase or decrease in temperature T.

The described stepwise selective deposition of a peptide in the presence of at least one further peptide is a preferred embodiment of the method according to the invention, wherein the mixing of the peptide solution/suspension with crystallization agent and the crystallization itself take place according to the invention in separate vessels or vessel sections.

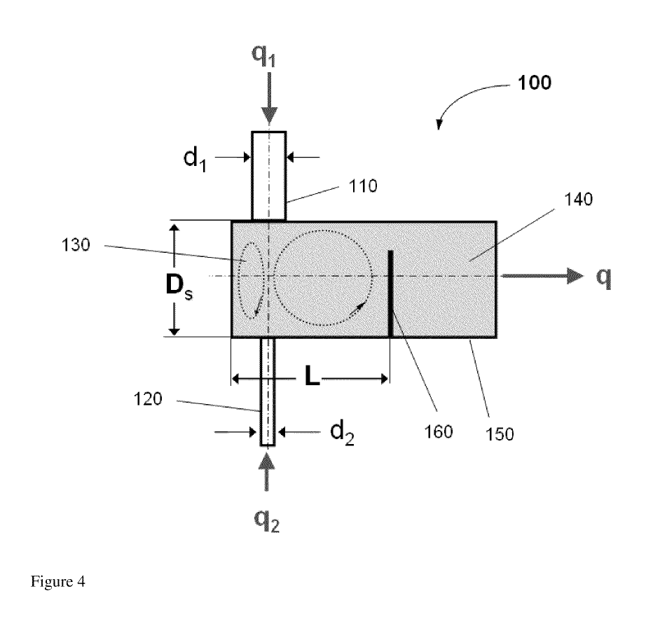

In FIG. 4, a preferred mixing element for step a) of the method according to the invention is illustrated schematically. The figure shows a cross-section of a jet mixer 100. This mixer comprises two inlets 110, 120 for the peptide solution (stream q₁) and the crystallization agent (stream q₂). The diameters of the inlets are d₁ and d₂. The jet mixer preferably has a tubular design having a diameter Dₛ. The ratio d₁/Dₛ is preferably in the range of 0.1≦d₁/Dₛ≦0.4, especially preferably in the range of 0.2≦d₁/Dₛ≦0.3. The ratio d₂/Dₛ is preferably in the range of 0.05≦d₂/Dₛ≦0.3, especially preferably in the range of 0.08≦d₂/Dₛ≦0.13.

Within the jet mixer is the mixing chamber 150, which is divided by an orifice plate 160 into a mixing zone 130 and an outlet zone 140. The volume of the mixing zone is preferably about ¾ of the mixing chamber volume, the volume of the outlet zone accordingly ¼ of the mixing chamber volume. As indicated by arrows in FIG. 130, there is a prevalence in the mixing zone of a macroscopic convection having high turbulence which is caused by the clashing streams q₁ and q₂. In contrast, the stream in the outlet zone ranges from being far less turbulent to being not turbulent at all. The mixture of peptide solution and crystallization agent is added to a heat exchanger and/or a vessel/vessel section for crystallization via the outlet of the jet mixer (stream q).

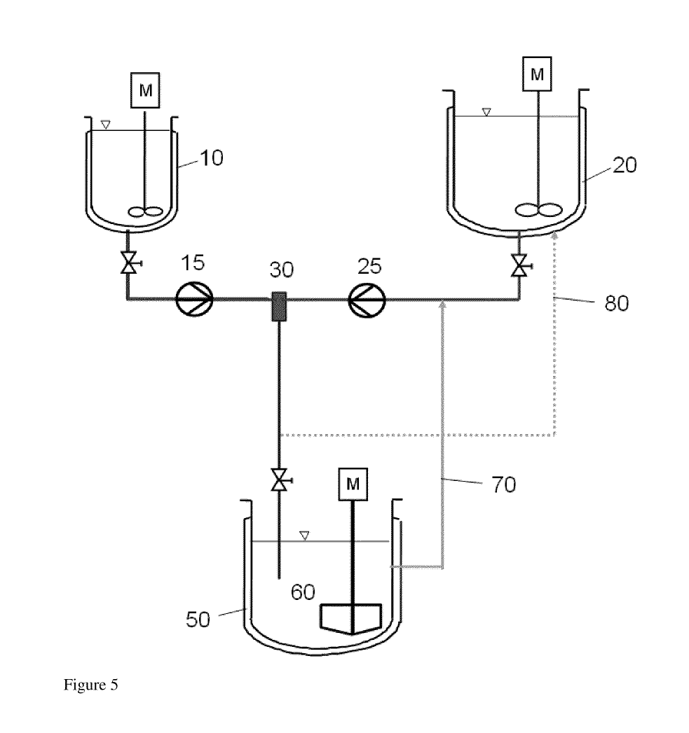

FIG. 5 shows a preferred embodiment of a device for carrying out the method according to the invention. The device comprises a vessel 10 for receiving crystallization agent, a vessel 20 for receiving peptide solution, a mixing element 30 which is connected to the vessel 10 via a pump 15 and to the vessel 20 via a pump 25, and a vessel 50 for crystallization which is connected to the mixing element 30. In a preferred embodiment, vessel 50 is connected to the connection between the vessel 20 and the mixing element 30 via a connection 70. This connection 70, which can have, e.g., a tubular design, allows (continuous) withdrawal of crystallization suspension from the vessel 50 and the addition of this suspension to step a) of the method according to the invention, which is carried out in the mixing element 30.

Connection 70 makes possible a form of operation which is termed here fed-back mode 1: after an initial mixing of crystallization agent from the vessel 10 and peptide solution from the vessel 20 in the mixing element 30, the mixture in vessel 50 is left for a certain period of time for maturation of the initial crystals. In a second and optionally further steps, crystallization agent is mixed with suspension or supernatant solution from vessel 50, which is added to the mixing element via the line 70 together with crystallization agent. As a result, it is possible to specifically dose the amount of crystallization agent stepwise and at defined intervals. The amount of crystallization agent is thus not added at once, but stepwise. In the mixing element, intensive mixing of the suspension or supernatant solution from vessel 50 and crystallization agent from vessel 10 takes place. The described method according to fed-back mode 1 is a preferred embodiment of the method according to the invention.

In a further embodiment of the device for carrying out the method according to the invention, the connection between heat exchanger 40 and vessel 50 is additionally connected to vessel 20 via a connection 80. This connection 80, which can have a tubular design, allows (continuous) withdrawal of a mixture, which comes from the mixing element, into the vessel 20.

Connection 80 makes possible a form of operation which is termed here fed-back mode 2: in a first step, crystallization agent from vessel 10 and peptide solution from vessel 20 are mixed intensively in the mixing element 30 before the mixed material is added to the crystallization vessel 50. In a second step, the suspension or supernatant solution from vessel 50 is added to the mixing element 30 via line 70 together with crystallization agent from vessel 10. After intensive mixing and optional warming or cooling, the mixture is conducted into the empty vessel 20 via the connection 80. In a third step, the mixing of the content of the vessel 20 with further crystallization agent and introduction of the mixture into vessel 50 take place. The second and third steps are optionally repeated one or more times. This approach has the advantage that crystallization agent is added uniformly and at the same time to a solution.

The volume of the vessel 50 is greater than the sum of the volumes of mixing element and the connections between the mixing element and vessel 50. When the suspension or supernatant solution from the vessel 50 in fed-back mode 1 is fed back into the vessel 50 via the connection 70 and the mixing element 30, it is mixed in vessel 50, more particularly at the inlet site in vessel 50 with suspension which has not been fed back yet. As a result, the feedback in feedback mode 1 may result in concentration fluctuations in the vessel 50. These fluctuations can disadvantageously affect the product quality. Such concentration fluctuations are avoided in fed-back mode 2.

In fed-back mode 2, the method according to the invention for depositing and/or recovering a peptide can take place more closely along the solubility curve than in fed-back mode 1. The described method according to fed-back mode 2 is an especially preferred embodiment of the method according to the invention.

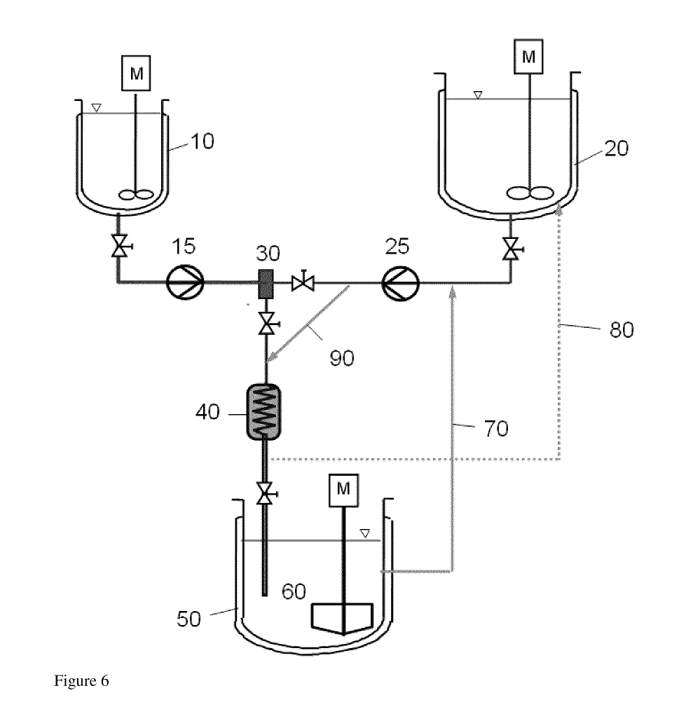

FIG. 6 shows a variant of the device shown in FIG. 5 for performing the method according to the invention. In addition to the elements already presented in FIG. 5, a heat exchanger 40 and a connection 90 are also present. When crystallizing purely by cooling or warming, where the crystallization is achieved solely by cooling down or warming the peptide solution, a mixing element can be dispensed with. In this case, the peptide solution/suspension from the vessel 50 in fed-back mode 1 is fed back into the vessel 50 again via the connection 70, the connection 90, and the heat exchanger 40. In the heat exchanger, the stepwise cooling down or warming of the peptide solution/suspension takes place in order to achieve a controlled crystallization. As already explained in the description for FIG. 5, the volume of the vessel 50 is greater than the sum of the volumes of the connections 70, 90 and the heat exchanger, so optionally cooled or warmed solution/suspension is fed back into the vessel 50 and meets here suspension of a different temperature which has not been fed back. In this case, this can result in temperature fluctuations which negatively influence the product quality. Here, fed-back mode 2 provides corrective action in which suspension/solution from vessel 50 is added to the heat exchanger via connection 70 in order to adjust the temperature and is, from this heat exchanger, added to the empty vessel 20 via connection 80. From the vessel 20, the solution is then added to the heat exchanger via the line 90 to adjust the temperature again and subsequently arrives back at vessel 50. The process can be, as needed, repeated one or more times. The method described here is a preferred embodiment of the method according to the invention.

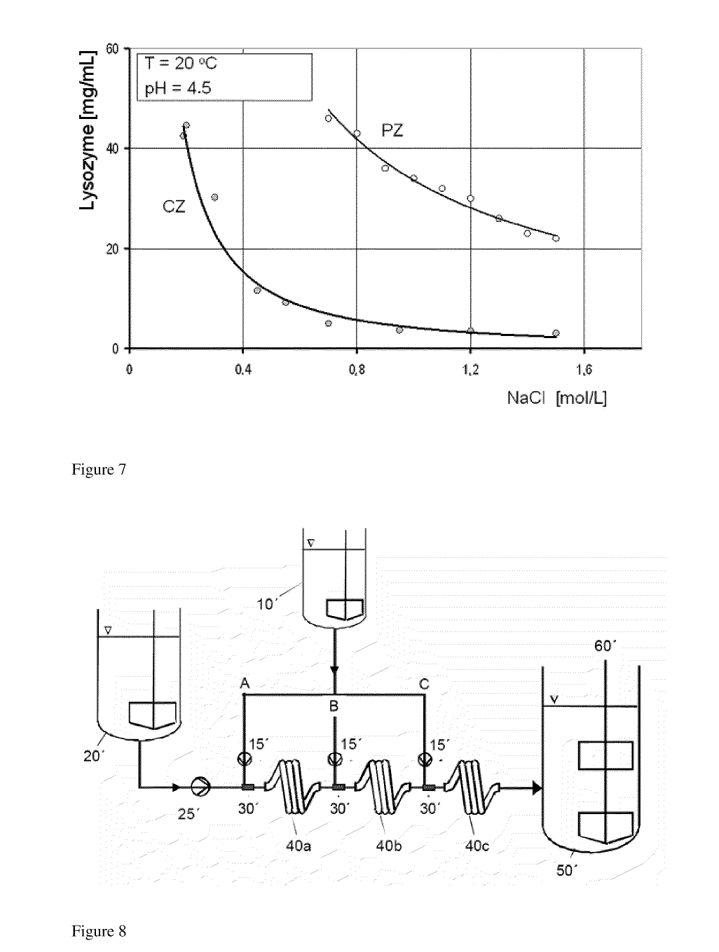

FIG. 7 shows schematically the solubility ratios of an aqueous lysozyme solution. The concentration of lysozyme is plotted against the concentration of crystallization agent NaCl. At a pH of 4.5 and a temperature of 20°, a lysozyme solution shows a range of supersaturation which is between the curves CZ and PZ. If, under the conditions mentioned, a NaCl concentration lying between the curves CZ and PZ is set, then the lysozyme slowly crystallizes. If the concentration of NaCl is raised and reaches the area to the right of the curve PZ, then the lysozyme is rapidly brought out of solution in the form of precipitate.

FIG. 8 shows a further embodiment of a device for carrying out the method according to the invention.

The device comprises a first container 10′ for receiving a crystallization agent, a second container 20′ for receiving a peptide solution, and a third container 50′ for crystallization which is stirred by means of a double-blade stirrer 60′. The containers 20′ and 10′ are connected to the container 50′ via low-shear pumps 15′, mixing elements 30′ which preferably have a jet-mixer design, and helically coiled tubular reactors 40a, 40b, and 40c. Such a device allows the stepwise crystallization of a peptide along the solubility curve. In a first step, peptide solution and a portion of the crystallization agent from the containers 20′ and 10′ are mixed in the mixing element 30′ between container 20′ and container 10′. The mixture passes into the reactor 40a. In the tubular reactor 40a, initial peptide agglomerates form under very uniform conditions. In the mixing element 30′ between reactor 40a and 40b, the suspension from reactor 40a is mixed with further crystallization agent from the container 10′. The mixture passes into the reactor 40b. In the tubular reactor 40b, further peptide agglomerates form and/or existing agglomerates grow under very uniform conditions. In the mixing element 30′ between reactor 40b and 40c, the suspension from reactor 40b is mixed with further crystallization agent from the container 10′. The mixture passes into the reactor 40c. In the tubular reactor 40c, further peptide agglomerates form and/or existing agglomerates grow under very uniform conditions. The suspension from reactor 40c passes into the container 50′, in which the crystallization is brought to an end under controlled conditions.

The tubular reactors 40a, 40b, and 40c can also act as heat exchangers and, e.g., absorb heat from crystallization or add heat to solution/suspension. The described method is a preferred embodiment of the method according to the invention.

The method according to the invention is not restricted to the methods described here. Further variants which arise, e.g., from the combination of the methods described here are also possible.

Through the method according to the invention, one or more advantages are achievable in a multiplicity of applications:
a reduction of concentration fluctuations and also of mechanical stresses on the particles,
the possibility of specific setting of saturation conditions and the avoidance of a supersaturation,
a selective crystallization and hence a better separation of various peptides in a solution which comprises more than one variety of peptide,
a uniform product having defined properties and the avoidance of polymorphous compounds,
the possibility to obtain fine crystals having a narrow particle size distribution,
a reduced section of damaged peptides,
a shortened processing time,
higher yields and a higher quality of deposited particles,
a simple scale-up.

### EXAMPLES

### Example 1

This example describes the crystallization of lysozyme. The crystallization was performed in a device according to FIG. 5. An aqueous NaCl solution having a concentration of 4.7 mol/L was introduced into vessel 10 as a crystallization agent. Lysozyme was likewise present in aqueous solution at a concentration of 20 g/L (vessel 20).

A 50-liter vessel (50) was used for the crystallization. Low-shear pumps (e.g., peristaltic pump: Watson Marlow) were used for the delivery of the solutions and suspensions. A jet mixer according to FIG. 4 was used, having two inlets having the diameters d₁=2.5 mm and d₂=6 mm. The tubular mixing chamber had a diameter of 24 mm. The jet mixer was operated turbulently with a Reynolds number in the region of Re=1500. The mixing time was 65 ms which. The pH of the mixture was 4.5, the mixing temperature was 20° C.

The diameter of the crystallization vessel was D=406 mm and was equipped with a blade stirrer, of which the blades had a ratio of height to diameter of h/d=0.5. In total, the stirrer carried 6 blades, having a ratio of blade diameter to the diameter of the crystallization vessel of d/D=0.55. The relative distance between stirrer and vessel was e/D=0.025.

The supply of the mixture of crystallization agent and peptide solution to the vessel 50 was conducted via a probe which was guided to almost the bottom of the vessel. The probe had a conical (funnel-shaped) angle of about 5°. The power output of the jet introduced into the vessel was less than 30 W/m³.

In FIG. 7, the result of two modes of operation are shown. Curve PZ shows the concentration progression of lysozyme in the solution as a result of the addition of large amounts (excess) of NaCl solution. The circles on the curve PZ show actual measured values. The lysozyme brought stepwise out of solution as precipitate was polymorphous and difficult to filter.

Curve CZ shows the progression upon addition of lower amounts of NaCl solution. The circles on the curve CZ show actual measured values which were obtained according to an approach according to feedback mode 2 (see description for FIG. 5). The lysozyme deposited stepwise in the form of crystals was of a greater purity, showed a narrower particle size distribution, and was easier to filter than the precipitate. Also, the yield of pure lysozyme when crystallizing was greater than when precipitating.

### REFERENCE SYMBOLS

10, 10′ Receiver container/vessel for crystallization agent
15, 15′ Pump
20, 20′ Receiver container/vessel for peptide solution
25, 25′ Pump
30, 30′ Mixing element in which step a) of the method according to the invention is carried out
40 Heat exchanger
40′ Tubular reactor
50, 50′ Vessel/container in which step c) of the method according to the invention is carried out
60, 60′ Stirrer
70, 80, 90 Connections
100 Mixing element, jet mixer
110, 120 Inlet
130 Mixing zone
140 Outlet zone
150 Mixing chamber
160 Orifice plate

Furthermore, the drawings show:
M=Stirring drive
T1=Temperature 1
T2=Temperature 2
FIC=Volume stream regulation
TIC=Temperature regulation

1. A method for depositing and/or selectively recovering a peptide/protein from a solution, comprising at least the following steps:
a) mixing a protein/peptide solution with a crystallization agent,
b) optionally cooling or warming,
c) crystallizing a protein/peptide,
wherein steps a) to c) proceed spatially separated from one another.

2. The method as claimed in claim 1, wherein step a) is performed in a jet mixer comprising at least two inlets and an orifice plate, between which is a mixing zone.

3. The method as claimed in claim 1 wherein the average mixing speed in step a) is in the range of 0.05 m/s≦v≦5 m/s.

4. The method as claimed in claim 1, wherein the pressure drop in step a) is in the range of 0.05 bar≦Δp≦20 bar.

5. The method as claimed in claim 1, wherein the macroscopic mixing time in step a) is in the range of 1 ms≦t_{Ms}≦1000 ms.

6. The method as claimed in claim 5, wherein the macroscopic mixing time in step a) is in the range of 8 ms≦t_{Ms}≦120 ms.

7. The method as claimed in claim 1, wherein step c) is performed with continuous stirring with a blade stirrer.

8. The method as claimed in claim 7, wherein the stirring speed is close to the point at which the crystals are just suspending.

9. The method as claimed in claim 7 wherein the stirrer is arranged with a relative eccentricity in the range of 0≦e/D≦0.035.

10. The method as claimed in claim 7, wherein the ratio between diameter d of the blade stirrer to the diameter D of the vessel or vessel section in which step c) is performed is in the range of 0.4≦d/D≦0.7.

11. The method as claimed in claim 1, wherein the ratio of volume of the vessel or vessel section in which step a) is carried out to the volume of the vessel or vessel section in which step c) is carried out is from greater than or equal to 0.02 and smaller than or equal to 0.08.

12. The method as claimed in claim 1, wherein a subsequent step d) is carried out between steps a) and c) or a), b), and c):
d) adding a portion of the solution of the crystallization suspension from step c) to the mixture in step a) or to the mixture in step b).

13. The method as claimed in claim 1, wherein the following steps a₁) and a₂) are carried out after steps a) and c) or a), b), and c):
a₁) admixing further crystallization agent,
a₂) optionally repeating steps a₁) and a₂).
